(19) United States
(12) Patent Application Publication
(10) Pub. No.: US 2022/0342197 A1
(43) Pub. Date: Oct. 27, 2022

(54) SUBASSEMBLY, OBJECTIVE AND LONG THING OPTICAL IMAGE TRANSFER SYSTEM

(71) Applicant: FISBA AG, St. Gallen (CH)

(72) Inventors: Romedi SELM, Wittenbach (CH); Martin FORRER, Appenzell (CH)

(21) Appl. No.: 17/636,584

(22) PCT Filed: Jun. 23, 2020
(86) PCT No.: PCT/EP2020/067428

§ 371 Date: Feb. 18, 2022

(30) Foreign Application Priority Data
Aug. 20, 2019 (EP) 19192423.2

(51) Int. Cl. G02B 23/24; A61B 1/00; G02B 7/18

(52) U.S. Cl. CPC G02B 23/243; A61B 1/00096; A61B 1/00179; A61B 1/0011; G02B 7/1805

(57) ABSTRACT

The invention relates to a subassembly (1) for an objective (10), in particular for a long and slim optical image transmission system, for example an endoscope, an objective (10) and an optical image transmission system, as well as methods for manufacturing a subassembly and an objective. The subassembly (1) comprises a prism (2) and an aperture element (3), wherein the prism (2) and the aperture element (3) are fixedly connected to each other, preferably by means of a putty or via direct bonding.

The invention relates to a sub-assembly for an objective, in particular for a long and slim optical image transmission system, for example an endoscope, an objective and an optical image transmission system, and methods for manufacturing a subassembly and an objective.

Long and slender optical image transmission systems, for example, endoscopes, are widely used to examine and diagnose intracavitary organs, intraorganic wall surfaces, or other targets by inserting a tubular insert into a body cavity. Endoscopes are also used in the industrial field to inspect the inside of pipes, boilers, machinery, chemical plants, etc.

Such image transmission systems typically include an optical illumination system for emitting illumination light at the distal side of the image transmission system and an eyepiece at the proximal side of the image transmission system, i.e., at the viewer's side.

Such an image transmission system may have a flexible or a rigid tubular housing.

Simple image transmission systems are realized with a straight field of view. That is, the axis of the field of view is in the direction of the mechanical axis of the tubular housing and the field of view is limited by the shape of the housing and the optics in the housing. However, many applications require an enlarged field of view.

Preferably, in this case, the image transmission system has an angled objective at the distal end so that the optical axis of the distal opening makes an angle with the longitudinal axis of the image transmission system.

For an image transmission system with a flexible, tubular housing, the longitudinal axis is understood to be the direction of the stretched housing.

For the deflection of a light beam in the angled objective, a prism is usually provided.

An objective with a compound prism is known from U.S. Pat. No. 4,138,192, in which the actual deflecting prism is supported by two prisms arranged on both sides, the prisms being cemented to each other. Such objectives are complex and therefore expensive to manufacture.

U.S. Pat. No. 4,850,342 shows objectives with single prisms. In these cases, it is costly to create a mechanical connection.

U.S. Pat. No. 5,519,532 shows an objective with a single prism made of plastic. A sufficient optical imaging quality with precise reflection surfaces is difficult to achieve.

It is the task of the invention to present a sub-assembly, an objective, an optical image transmission system, as well as a method for manufacturing a sub-assembly and an objective, which avoid the disadvantages of the known and, in particular, allow easy assembly and good manageability with precise imaging properties.

The task is solved by the features of the independent claims.

A subassembly for an objective, in particular for a long and slim optical image transmission system, such as an endoscope, comprises a prism and an aperture element. The prism and the aperture element are fixedly connected to each other. Preferably, the prism is directly connected to the aperture element, with connecting means, for example an optical putty, being provided directly between the prism and the aperture element at most.

Preferably, the prism is connected to the aperture element via a putty or via direct bonding, such as hydrophilic bonding or laser-assisted bonding.

In putty bonding, components with polished surfaces, preferably components made of glass, are joined by bonding the adjacent surfaces together with a thin, optically transparent putty layer. In most cases, a UV-curing optical putty is used, which has a refractive index similar to that of the components to be joined. For good optical performance, the size of the mastic gap must be considered and taken into account in the optical design.

In bonding, the optical putty is dispensed with and the adjacent optical surfaces are brought together directly. With this process, even better optical results can be achieved. However, the optical surfaces must be manufactured very precisely.

For example, the adhesive force can be generated by adhesion (so-called gating). The bond can be sensitive to temperature fluctuations.

In direct bonding, hydrophilic or hydrophobic surfaces are brought together under high temperature and pressure. The adhesion is based on the Van der Waals interaction. As a rule, a very stable bond can be achieved.

In anodic bonding, an additional electrical voltage is applied.

In particular, the prism and the aperture element are connected in such a way that the connection cannot be detached non-destructively or can only be detached with great effort, the connecting means being destroyed, for example, if an optical putty is removed with solvent.

The subassembly forms a one-piece component that can be easily mounted in a housing or socket. During assembly, the relative alignment between the prism and the aperture element is maintained. It is sufficient to align one of the components with respect to the mount or housing. It is also only necessary to attach either prism or aperture element to the mount or housing.

The attachment may be to the side surfaces of the aperture element. The subassembly can be designed so that the aperture element has a larger diameter than the prism transverse to the light transmission direction.

The aperture element may have a cuboidal basic shape with a height of typically about 0.5 mm to 1.5 mm, for example 1 mm, a width of typically about 0.5 mm to 5 mm, for example 1 mm, and a length of typically 1 mm-3 mm, for example about 3 mm. The exit surface of the prism may have the same width as the width of the aperture element, but a shorter length.

In the assembled state, the aperture element is at least partially in contact with the housing or socket, for example with the smaller side surfaces of a cuboidal base shape. On the other hand, the prism can stand freely in space and not touch the housing. The mirror surfaces need not necessarily be coated, since the reflections can be due to total internal reflection at the prism-air interface.

In particular, the prism has a beam entrance surface and a beam exit surface. The beam exit surface preferably abuts the aperture element. The beam entrance surface and a beam exit surface enclose an angle γ.

This angle corresponds to the angle by which an incident beam is to be deflected by the prism.

The beam entrance surface and/or the beam exit surface can have an area of 0.5 mm×0.5 mm to 3 mm×3 mm, for example about 1 mm×1 mm.

The angle is preferably in a range of 10° to 80°, further preferably between about 20° and 45°, for example 22.5°.

Preferably, the prism has two reflecting surfaces on which light entering through the beam entrance surface is reflected.

In this case, the condition θ>θc must be satisfied for all incident rays at the interface, where θc is the critical angle at which the ray just does not emerge:

$$θc = arcsin ( \frac{n 2}{n 1} ) .$$

Here, n2 is the refractive index of the thinner medium (for example, air) and n1 is the refractive index of the prism glass.

The deflection angle γ and the above condition thus provide a measure of the necessary refractive index of the prism glass.

If no glass fulfilling this condition is available for a desired deflection angle, the reflection surfaces must be provided with a mirror layer. A dielectric layer is preferred for this purpose. Alternatively, metal layers or a combination of metal layer and dielectric layer are also possible.

With a coating, reflection is also possible at angles at which total internal reflection no longer occurs.

In an advantageous embodiment of the subassembly, the prism and/or the aperture element are made of glass.

With glass-fabricated components, very flat surfaces, especially mirror surfaces, can be realized and thus a high imaging quality is made possible.

In particular, the prism has polished glass surfaces.

Mirroring of the reflecting surfaces is not absolutely necessary.

The task is further solved by a objective, in particular for a long and slim optical image transmission system, for example an endoscope. The objective comprises at least one subassembly as described above.

In particular, the sub-assembly is mounted in a cylindrical housing such that the exit surface of the prism is arranged perpendicular to the longitudinal axis of the housing.

In a preferred embodiment of the objective, the objective comprises an, preferably plano-concave, entrance lens and/or at least one imaging lens.

The subassembly and the lenses are arranged in a common, in particular cylindrical, housing.

The optical axis of the entrance lens is arranged in particular perpendicular to the entrance surface of the prism.

The objective thus has an oblique viewing angle, since the optical axis of the entrance lens is inclined with respect to the longitudinal axis of the housing.

In particular, the optical axis of the imaging lens is arranged perpendicular to the exit surface of the prism. The objective preferably has three imaging lenses whose optical axes are aligned with each other.

A tuning ring can be arranged in the objective between the entrance lens and the subassembly. The tuning ring can be used to compensate for imaging errors that can occur, for example, because the reflective surfaces of the prism are spaced at an unsuitable distance. A light beam passing centrally through the entrance lens then does not hit the center of the imaging lens. A centering error can lead to imaging errors.

By varying the distance between the entrance lens and the subassembly, a centering can be achieved which is necessary for a required imaging quality. With the tuning ring, the appropriate distance for optimal centering can be determined. Precise centering can also be achieved by shifting the entrance lens, even without a tuning ring.

The task is further solved by an image transmission system, for example an endoscope, with at least one objective as described above. In particular, the objective may be detachably attached to a system housing. The objective can be replaced, maintained and/or cleaned.

An image transmission system may also have a system housing to which two objectives are attached, so that two image transmission channels are used. With two image transmission channels, three-dimensional representations can be realized, for example.

The image transmission system can be assembled from a modular system, which can include several objectives, for example with different viewing angle inclinations, each of which can be selectively connected to the system housing.

The problem is further solved by a method of manufacturing a subassembly as described above, wherein a prism having a beam entrance surface and a beam exit surface is fixed to an aperture element in such a way that the beam exit surface abuts the aperture element and covers the aperture opening. The fixation is performed in such a way that the prism is firmly connected to the aperture element.

In particular, a connection is made which cannot be detached non-destructively or can be detached only with great effort, the connecting means being destroyed, for example, if, for example, an optical putty with solvent is removed.

Advantageously, the prism is putty to the aperture element. For example, NOA61 (Norland Optical Adhesive 61) can be used as the optical cementing agent.

A connection can also be made by hydrophilic bonding. Hydrophilic bonding without putty is a mineral bonding technique, especially for glasses and transparent crystalline materials. This bonding technique guarantees full transparency at the joint, high mechanical and thermal stability and allows precise alignment of the interfaces.

In an advantageous embodiment of the process, an aperture wafer is provided. This preferably has an aperture substrate coated with black chromium. The substrate may be glass, for example D263T thin glass or quartz glass.

The aperture wafer has at least one aperture opening, but preferably an array of a plurality of aperture openings.

Outside the apertures, the wafer is light-impermeable. The aperture substrate, for example made of glass, may be coated with black chrome for this purpose.

In addition, at least one prism base having a beam entrance surface and a beam exit surface is provided.

The aperture wafer may have a thickness of between 0.5 mm and 1.5 mm, preferably of about 0.7 mm.

The area of the aperture wafer may be between 10 mm×10 mm and 100 mm×100 mm, preferably about 45 mm×45 mm.

The aperture diameter can be between 0.1 mm and 1 mm, preferably about 0.4 mm. The coating preferably has an optical density greater than 3.

Typically, one prism base body is provided for each aperture or for a series of apertures of the aperture wafer. The prism base body is preferably rod-shaped and has a length of 10 mm-100 mm, for example 40 mm.

The aperture wafer and the prism base body are connected, with one prism base body each being placed on an aperture opening or on a row of aperture openings.

The putty gap thickness can be controlled with microbeads, thin filaments, thin films, coating, for example vapor deposition, or with a spacer tool.

The putty gap thickness is between 0.01 mm and 0.05 mm, for example 0.03 mm.

Then the aperture wafer is cut, for example with a wafer saw, so that one or more individual subassemblies are obtained. If necessary, a prism base body is also cut during the cutting process to cover a series of aperture openings.

The aperture wafer can be equipped with auxiliary lines that facilitate positioning of the prism base body and specify the parting lines for cutting the aperture wafer. Alternatively and/or additionally, markings may be provided to facilitate positioning of, for example, a cutting machine. Auxiliary lines and/or markings may be scribed into the coating or may be produced lithographically.

The task is further solved by a method for mounting a objective as described above, wherein a subassembly is provided, preferably in a method as described above, and the subassembly is fixed, in particular glued, into a housing.

When assembling the objective, an entrance lens may first be fixed to the housing, and preferably a tuning ring may be positioned between the entrance lens and the subassembly. The tuning ring determines the distance between the entrance lens and the subassembly so that, if possible, no centering error occurs.

It is also possible to first attach the subassembly to the housing, then move an entrance lens opposite the subassembly until an optimal position is found. Then the entrance lens is fixed to the housing.

During the shifting, it is possible to check whether there is a centering error. The best position of the entrance lens can be set for optimum imaging quality.

Advantageously, for the bonding of the subassembly in a housing, adhesive is introduced through an opening in the housing into the space between the subassembly and the housing, and the opening is closed again by the adhesive.

In an advantageous embodiment of the process, the subassembly is pushed into the housing by means of an insertion tool. The insertion tool has a mounting head with a mounting recess for receiving the orifice element.

The mounting recess can be in the form of a groove in which a panel element with a cuboid basic shape can be accommodated.

The insertion tool also has a guide body whose outer diameter corresponds to the inner diameter of the housing, so that the insertion tool can be inserted into the housing without tilting. The orientation of the subassembly in the housing is therefore predetermined by the mounting head, or the direction of the mounting recess, and very precise positioning can be achieved.

The insertion tool may have a guide pin that points radially away from the guide body of the insertion tool and indicates the orientation of the subassembly, particularly the prism. Preferably, the guide pin engages a slot provided in the housing when the subassembly is in the correct orientation with respect to the housing and the inclination of the entrance lens.

The invention further relates to an insertion tool for inserting a sub-assembly into a housing in a process for mounting an objective as described above, the insertion tool comprising a mounting head having a mounting recess for receiving the subassembly and a guide body having an outer diameter corresponding to the inner diameter of the housing. Preferably, the insertion tool has a guide pin that indicates the orientation of the subassembly and can cooperate with a slot in housing.

Preferred embodiments of the invention are explained in more detail in the following description with reference to the accompanying drawings. In this connection, corresponding elements are provided with matching reference signs.

It shows

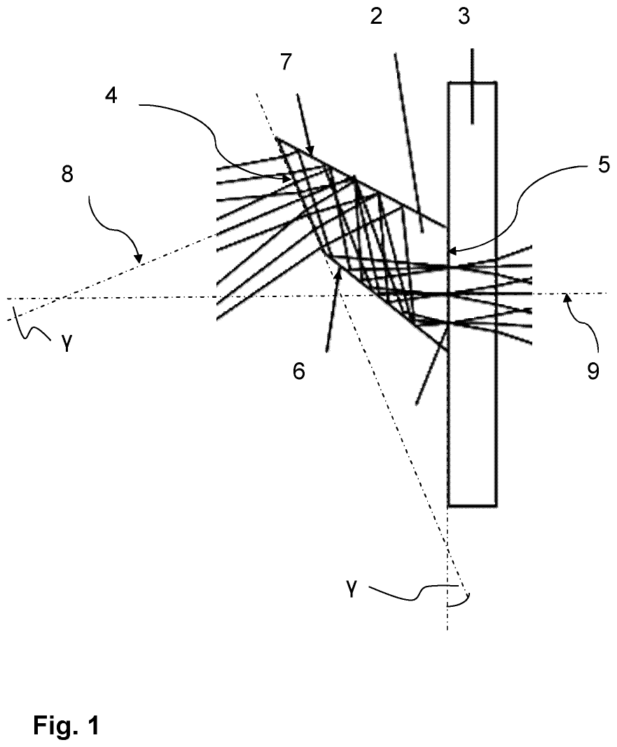

FIG. 1 a subassembly in lateral sectional view;

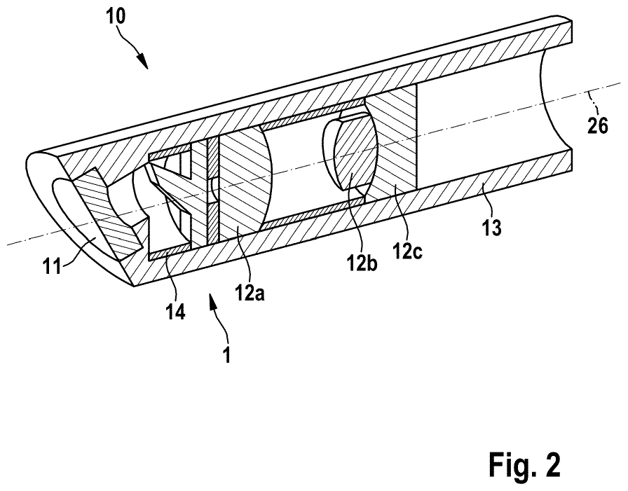

FIG. 2 an objective in perspective sectional view;

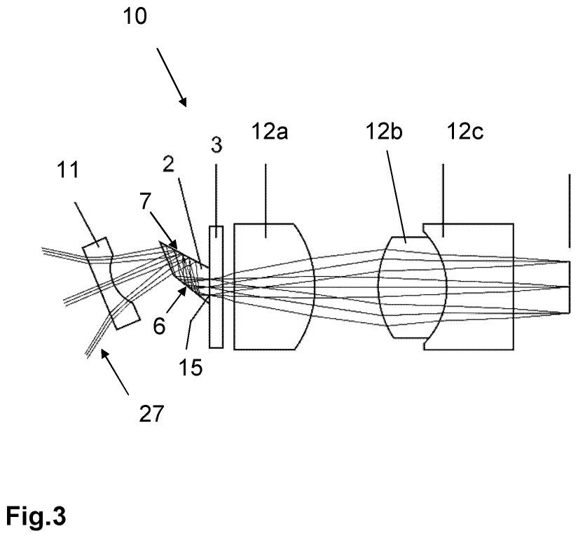

FIG. 3 a schematic representation of a beam path in an objective in lateral sectional view;

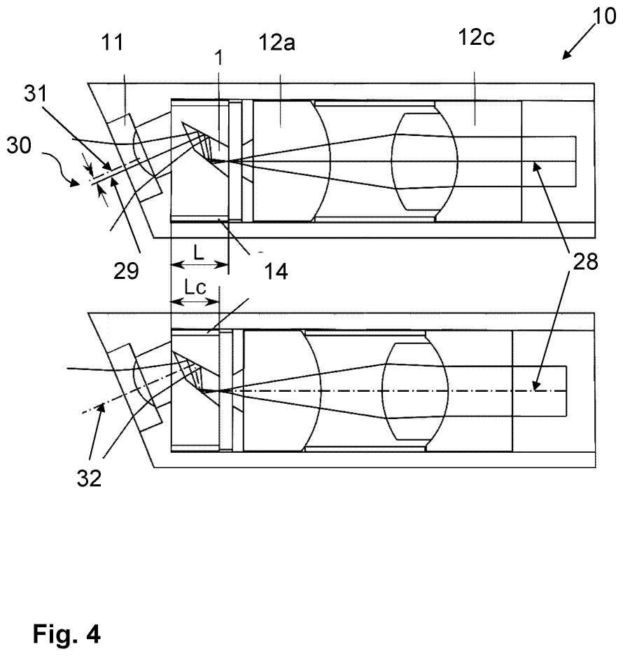

FIG. 4 a schematic representation of objectives in comparison in lateral sectional view;

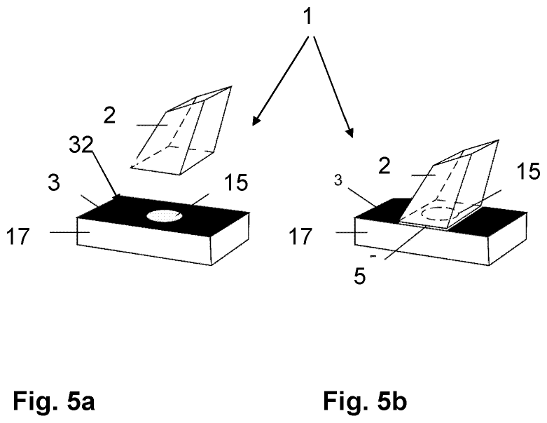

FIGS. 5a, 5b subassemblies in perspective view;

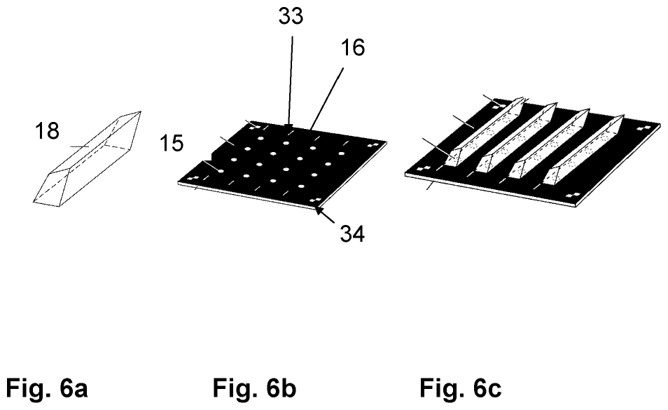

FIGS. 6a-6b schematic illustrations of manufacturing steps;

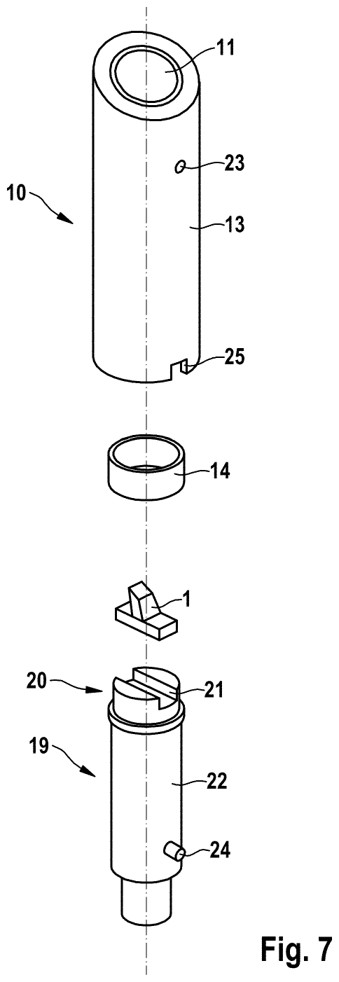

FIG. 7 an exploded view of an objective and an insertion tool.

FIG. 1 shows a subassembly 1 in lateral sectional view. The subassembly 1 comprises a prism 2 and an aperture element 3, which are firmly connected to each other, preferably via a putty or direct bonding.

The prism 2 has a beam entrance surface 4 and a beam exit surface 5, with the beam exit surface 5 abutting the aperture element 3.

The beam entrance surface 4 and the beam exit surface 5 enclose an angle γ corresponding to the angle γ between the direction 8 of an incident beam and the direction 9 of an outgoing beam.

The prism 2 has two reflection surfaces 6, 7. These can be mirror-coated.

FIG. 2 shows a objective 10 in perspective sectional view. The objective 10 comprises a subassembly 1, a plano-concave entrance lens 11 and three imaging lenses 12a, 12b, 12c, which are arranged in a housing 13.

The concave surface of the entrance lens 11 is not perpendicular to the longitudinal axis 26 of the housing 13, so that the objective 10 is angled.

FIG. 3 shows a schematic diagram of a beam path in an objective 10 in a side sectional view.

The beam 27 passes, for example with a field angle of ±35°, through the plano-concave entrance lens 11. The beam is then passed through the prism 2, whereby a total reflection takes place at the reflection surfaces 6, 7, whereby the beam is deflected. The beam then passes through the aperture 15 of the aperture element 3. Subsequently, the beam passes through three imaging lenses 12a, 12b and 12c.

FIG. 4 shows a schematic representation of objectives 10 in comparison in lateral sectional view.

In the objective 10 in the upper illustration, the entrance lens 11 and the subassembly 1 are arranged relative to each other such that a centrally imaged light beam 28 corresponds to an incoming light beam 29 that has an offset 30 from the central axis 31 of the entrance lens 11.

A tuning ring 14 of length L is arranged in the objective 10 of the upper illustration.

When this is replaced by a tuning ring 14 of length Lc, as shown in the lower illustration, the distance between the entrance lens 11 and subassembly 1 is altered such that a centrally incoming beam 32 is imaged as a centrally outgoing beam 28.

FIGS. 5a, 5b show subassemblies 1 in perspective view. The subassembly comprises a prism 2 and an aperture element 3.

The aperture element 3 has an aperture opening 15.

An opaque coating 32 is applied to an aperture substrate 17.

After assembly, the beam exit surface 5 of the prism 2 lies firmly against the aperture element 3 and covers the diaphragm opening 15.

FIGS. 6a-6b show schematic illustrations of manufacturing steps. First, at least one prism base body 18 is provided according to FIG. 6a.

Further, an aperture wafer 16 having a plurality of aperture openings 15 is provided.

Auxiliary lines 33 and markings 34 are provided on the aperture wafer 16.

The auxiliary lines help to arrange the prism bases 18 on the aperture wafer 16 in such a way that each row of apertures 15 is evenly covered by a prism base 18.

The prism bases 18 are firmly connected to the diaphragm wafer 16.

Subsequently, the aperture wafer 16 can be cut parallel to the rod-shaped prism base bodies 18, with the markings 34 serving as orientation.

Finally, the prism bases are cut together with the aperture wafer 16 to obtain individual subassemblies 1 as shown in FIG. 5b.

FIG. 7 shows an exploded view of an objective 10 and an insertion tool 19.

The insertion tool 19 has a mounting head 20 with a mounting recess 21 for receiving the aperture element 3 and a guide body 22, the outer diameter of which corresponds to the inner diameter of the housing 13, so that the insertion tool 19 with the subassembly can be pushed into the housing 13 with a precise fit.

The insertion tool 19 has a guide pin 24 that points radially away from the guide body 22 of the insertion tool 19. The guide pin 24 can engage a slot 25 provided in the housing 13 when the subassembly 1 has the correct orientation with respect to the housing 13.

An entrance lens 11 is attached to the housing 13. A tuning ring 14 is positioned between the entrance lens 11 and the subassembly 1.

1.-18. (canceled)

19. A subassembly for an objective, the subassembly comprising a prism and a aperture element, wherein the prism and the aperture element are firmly connected to each other.

20. The subassembly according to claim 19, wherein the prism and the aperture element are firmly connected to each other by means of a putty or via a direct bonding.

21. The subassembly according to claim 19, wherein the prism has a beam entry surface and a beam exit surface and the beam exit surface abuts the aperture element, and wherein the beam entry surface and the beam exit surface enclose an angle γ.

22. The subassembly according to claim 21, wherein y is between 10° and 80°.

23. The subassembly according to claim 19, wherein the prism has two reflection surfaces which are not coated or which are mirror-coated.

24. The subassembly according to claim 19, wherein at least one of the prism and the aperture element are made of glass.

25. An objective, wherein the objective comprises at least one subassembly according to claim 19.

26. The objective according to claim 25, wherein the objective comprises at least on of an entrance lens and at least one imaging lens (12a, 12b, 12c), which are arranged in a common housing (13).

27. The objective according to claim 26, wherein the entrance lens is plano-concave.

28. The objective according to claim 25, wherein a tuning ring is arranged between the entrance lens and the subassembly.

29. An image transmission system, for example endoscope, comprising at least one objective according to claim 25.

30. The image transmission system according to claim 29, wherein the image transmissions system is an endoscope.

31. A method of manufacturing a subassembly according to claim 19, wherein a prism having a beam entry surface and a beam exit surface is fixed to an aperture element in such a way that the beam exit surface abuts the aperture element and covers the aperture opening, wherein the prism is fixedly connected to the aperture element.

32. The method according to claim 31, wherein the prism is adhered by putty to the aperture element.

33. The method according to claim 31, comprising the steps of,
providing an aperture wafer, having at least one aperture opening;
providing at least one prism base having a beam entrance surface and a beam exit surface;
joining the aperture wafer and the prism base body; and
cutting the aperture wafer so that a single subassembly is obtained.

34. The method according to claim 33, wherein the aperture wafer is a coated aperture substrate.

35. A method of assembling an objective according to claim 25, comprising:
manufacturing a subassembly wherein a prism having a beam entry surface and a beam exit surface is fixed to an aperture element in such a way that the beam exit surface abuts the aperture element and covers the aperture opening, wherein the prism is fixedly connected to the aperture element; and
fixing the subassembly in a housing.

36. The method according to claim 35, wherein first an entrance lens is fixed to the housing.

37. The method according to claim 36, wherein a tuning ring is positioned between the entrance lens and the subassembly.

38. The method according to claim 35, wherein first subassembly is fixed to the housing, then an entrance lens is shifted with respect to the subassembly until an optimal position is found and subsequently the entrance lens is fixed to the housing.

39. The method according to claim 35, wherein for bonding from the subassembly into a housing adhesive is brought through an opening in the housing between the subassembly and the housing.

40. The method according to claim 35, wherein the subassembly is pushed into the housing by means of an insertion tool, which has a mounting head with a mounting recess for receiving the aperture element, and a guide body whose outer diameter corresponds to the inner diameter of the housing.

41. The method according to claim 40, wherein a guide pin facing radially away from the guide body of the insertion tool engages a slot provided in the housing when the subassembly has the correct orientation with respect to said housing.

42. An insertion tool for inserting a subassembly into a housing in a method according to claim 35, wherein the insertion tool comprises a mounting head with a mounting recess for receiving the subassembly, and a guide body whose outer diameter corresponds to the inner diameter of the housing.
